(19) United States
(12) Patent Application Publication
(10) Pub. No.: US 2012/0237924 A1
(43) Pub. Date: Sep. 20, 2012

(54) Method to prevent missing PCR template and/or reaction mixture and its further use of preventing missing solution(s) in experiment

(76) Inventors: Fushan Wang, Broomall, PA (US); Weidong Zhang, Tampa, FL (US); Lixian Jiang, Wesley Chapel, FL (US)

(21) Appl. No.: 13/065,094

(22) Filed: Mar. 14, 2011

(51) Int. Cl. C12Q 1/68

(52) U.S. Cl. 435/6.1

(57) ABSTRACT

This invention discloses a method by colored solution to prevent missing a solution in experiment.

In an experiment, it is common for a technician to miss one step, and miss to add one solution into a mixture, especially when there are many specimens involved. For instances, there are nine specimens labeled 1, 2, 3 . . . 9 respectively. Each specimen needs to add solution A, B, C and D in sequence. After add solution A to all specimens, when solution B is needed, it may mistakenly skip specimen 3, and add solution B to the rest of specimens. The same thing may happen to other specimens or other solutions.

To prevent the similar mistake, one solution is to check the volume in each specimen after each step. Use the same illustration above, the specimen 3 with skipped step has less volume than other specimen. However, when each step only add a small volume, it is hard to check the volume with naked eye. Use the same case above, if the volume of solution B is only μl or less that μl, it seems unlikely to check the volume with the naked eyes. Therefore, it is need to explore another solution for this problem.

In this invention, the solutions are mixed with a dye, which pre-label the solution in a conspicuous different color. Thus, when one specimen miss one solution, it is easy to check the difference. Use the same illustration above, suppose that solution B is a red solution, while solution A is transparent or different color. When specimen 3 is skipped, it is easy to tell that solution B was not added into the mixture with a naked eye. This will alert technician possible problem.

It is essential that the color should not interference the experiment. Some experiments such as real-time PCR performance, the experiment plates are measured by plate reader which calculates the light adsorption of certain light range into actual amount. Therefore, the color for labeling should be in different range of light, and do not interference experiment measure.

In a specific example: for regular PCR on DNA template or reverse transcriptase PCR (RT-PCR) on RNA template or quantitative real time PCR on DNA template or quantitative real time reverse transcriptase (RT-PCR) on RNA template, a dye (will be disclosed in detail further when necessary) is added into the DNA samples or RNA samples after their final preparations. Missing of PCR DNA or RNA template in a tube or a well in a plate is notified by missing the color of the dye; after PCR reaction mixture is added a specific color change will develop which can be prominently recognized by naked eye so that missing of PCR reaction mixture in a PCR tube or well in a plate will be caught by a technician. Only both DNA or RNA template and PCR reaction mixture co-exist will the color change show up. The dye doe not interfere with PCR reaction and its peak does not comingle with dyes peaks of real time PCR labeling probes.

Cresol Red is such a dye which is yellow at pH 7.2 and red at pH 8.8. DNA or RNA samples with Cresol Red in Tris-EDTA buffer (pH 7.2) have yellow color. When PCR mixtures (pH 8.8) are added into the DNA or RNA samples with Cresol Red already in a PCR tube or well in a plate, the PCR tube or well in a plate will turn into red from yellow. Of note, PCR can be performed in buffer pH 8.8 in general and Cresol Red does not inhibit Taq polymerase as other common loading dyes.

1. A method of prevention missing a solution by colored solution consisting of the steps: a. prepares the dye in different solution; b. adds the mix solution into specimens; c. the color changed in specimen indicates the position of experiment.

2. The colored solution in claim 1 consisting of any dye that has color and do not interference experiment.
